(19) United States
(12) Patent Application Publication
(10) Pub. No.: US 2005/0235376 A1
(43) Pub. Date: Oct. 20, 2005

(54) Method for producing a transgenic plant having modified transport of substances

(76) Inventors: Wolf-Bernd Frommer, Stanford, CA (US); Mechthild Tegeder, Pullman, WA (US); Axel Hirner, Tubingen (DE); Wolfgang Koch, Kusterdingen-Wankheim (DE)

Correspondence Address: PENDORF & CUTLIFF, 5111 MEMORIAL HIGHWAY, TAMPA, FL, 33634-7356, US

(21) Appl. No.: 10/514,513

(22) PCT Filed: May 12, 2003
(86) PCT No.: PCT/DE03/01512

§ 371 Date: Jun. 15, 2005

(30) Foreign Application Priority Data
May 13, 2002 (DE) 102 21 224.4

(51) Int. Cl. C07H 21/04; A01H 1/00; C12N 15/82

(52) U.S. Cl. 800/278; 435/468; 536/23.6

(57) ABSTRACT

The invention relates to a method for producing a transgenic plant in which the capability for incorporating undesired nitrogen compounds, especially amino acids, in the storage organs is reduced compared with the wild type, comprising the steps: introducing at least one DNA sequence and/or at least one RNA sequence corresponding to the DNA sequence and/or a mixed sequence composed of DNA and RNA nucleotides corresponding to the DNA sequence with the coding region for an amino acid transporter or parts thereof into a plant cell, wherein the DNA and/or RNA sequence and/or mixed sequence is used in sense or antisense orientation and the expression of an endogenous amino acid transporter gene is prevented or reduced,
and regenerating a plant from this plant cell, wherein the DNA and/or RNA sequence is a sequence from Beta vulgaris.

### BACKGROUND OF THE INVENTION

Field of Invention

The invention relates to a method for producing a transgenic plant in which the capability for incorporating undesired nitrogen compounds, especially amino acids, in the harvest and/or reproductive organs is reduced compared with the wild type. The invention further relates to DNA sequences and RNA sequences corresponding to these, which contain the coding region for an amino acid transporter or parts thereof, their use as well as vectors, mobile genetic elements, bacteria, host cells, plant cells, plants, seeds and other reproductive material of plants which contain the DNA and/or RNA sequences.

Plants absorb nitrogen, which is required to build cell substance, predominantly in the form of nitrate, but also as ammonium and, to a small extent, as amino acids, for example via the roots. The nitrate is reduced to amino nitrogen and incorporated in organic compounds. This occurs predominantly in the leaves of the plant. Nitrogen-containing organic compounds, for example, amino acids are transported via a vascular system, the phloem, from meristematic species such as the leaves to consumptive tissue and organs, for example, reproductive organs or storage organs (roots, bulbs, beet etc.).

The transport processes are especially important during the transfer of nitrogen compounds in the course of the aging (senescence) of leaves, approximately towards the end of the vegetation period. In this situation, nitrogen compounds are transferred from the dying leaves into other organs, for example storage organs in order to thus keep the loss of cell material as low as possible.

The transport of amino acids is mediated by proteins, so-called amino acid transporters, to which the amino acid permeases (AAPs=amino acid permeases) belong. AAPs not only mediate the transport of a broad spectrum of amino acids with comparatively low selectivity but also of other organic nitrogen compounds, for examine amides such as glutamine and asparagine as well as citrulline, gamma amino butyric acid or auxine. These are therefore presumed to have a key role in the distribution of organic nitrogen compounds within the plant.

Amino acid transporter genes from Arabidopsis thaliana are known for example from EP 0652955. AAP genes from sugar beet (Beta vulgaris) on the other hand have not yet been isolated.

The incorporation of organic nitrogen compounds such as proteins and amino acids in harvest organs of plants is undesirable in many cases. The object of the present invention is thus to provide the possibility of producing plants in which the incorporation of organic nitrogen compounds in their harvest organs is reduced compared with the wild type.

This object is solved by a method which comprises the steps of introducing at least one DNA sequence and/or at least one RNA sequence corresponding to the DNA sequence and/or a mixed sequence composed of DNA and RNA nucleotides corresponding to the DNA sequence with the coding region for an amino acid transporter or parts thereof into a plant cell, wherein the DNA and/or RNA sequence and/or mixed sequence is used in sense or antisense orientation and the expression of an endogenous amino acid transporter gene is prevented or reduced, and regenerating a plant from this plant cell, wherein the DNA and/or RNA sequence is a sequence from Beta vulgaris.

Plants which were produced by the method according to the invention have a significantly reduced content of organic nitrogen compounds, especially amino acids, in their harvest organs. In a plant cell into which the DNA sequence and/or the RNA sequence corresponding to the DNA sequence and/or the RNA/DNA mixed sequence corresponding to the DNA sequence was introduced in antisense orientation, the translation of the mRNA of the endogenous amino acid transporter gene is hindered by attachment of an antisense RNA onto the mRNA. In the case where a DNA sequence is introduced, the antisense RNA is formed by transcription of the DNA sequence. In the case of the RNA sequence corresponding to the DNA sequence, this can represent the antisense RNA itself. A posttranscriptional gene silencing (PTGS=posttranscriptional gene silencing) of the endogenous amino acid transporter gene can also be brought about by co-suppression or RNA interference, by the DNA sequence or an RNA sequence corresponding to the DNA sequence being introduced in sense orientation into the plant cell, the RNA being used as double-stranded RNA in the case of RNA interference. In addition, it is also possible to use mixed sequences composed of RNA and DNA sequences, chimeric oligonucleotides, as has been described for example by Rice et al. (2000), Plant Physiology 123, 427-437, to bring about a gene silencing of the endogenous amino acid transporter gene. The plants produced by the method according to the invention are in this way largely prevented from forming amino acid transporters. By coupling to suitable tissue-specific promoters, it is also possible to specifically reduce or suppress the formation of amino acid transporters in certain tissue or organs of the plant, possibly in the leaves.

The term “parts” of a coding region used here designates nucleotide sequences with at least 20 nucleotides which make possible a repression of an amino acid transporter gene in antisense orientation.

The term “an RNA sequence corresponding to the DNA sequence” used here designates an RNA sequence which has the same sequence of purine and pyrimidine bases as a DNA sequence but instead of the base thymine in the DNA sequence, has the base uracil.

The term “a mixed sequence composed of DNA and RNA nucleotides corresponding to the DNA sequence” designates a nucleotide sequence which has the same sequence of purine and pyri\grave{m}idine bases as a DNA sequence but which contains both DNA nucleotides and also RNA nucleotides and wherein the RNA nucleotide has the base uracil instead of the base thymine in the DNA sequence. Chimeric oligonucleotides for example represent such mixed sequences.

“Antisense orientation” of a DNA sequence means here that a transcription of the DNA sequence results in an mRNA whose nucleotide sequence is complementary to the natural (endogenous) mRNA so that its translation is impeded or prevented. “Antisense orientation” of an RNA sequence means that the RNA sequence is complementary to an endogenous mRNA and its translation by attachment is impeded or prevented.

A DNA sequence used in the method according to the invention preferably comprises the nucleotide sequence of SEQ ID NO: 5 or a nucleotide sequence complementary to this nucleotide sequence or hybridises with the nucleotide sequence of SEQ ID NO: 5 or a nucleotide sequence complementary to the nucleotide sequence of SEQ ID NO: 5. The DNA sequence can also, alternatively or additionally, comprise the nucleotide sequence(s) of SEQ ID NO: 1, SEQ ID NO: 2, SEQ ID NO: 3, SEQ ID NO: 4, SEQ ID NO: 6, SEQ ID NO: 7 or SEQ ID NO: 8 or a nucleotide sequence complementary to this nucleotide sequence, or hybridise with the nucleotide sequence of SEQ ID NO: 1, SEQ ID NO: 2, SEQ ID NO: 3, SEQ ID NO: 4, SEQ ID NO: 6, SEQ ID NO: 7 or SEQ ID NO: 8 or a nucleotide sequence complementary to the nucleotide sequence of SEQ ID NO: 1, SEQ ID NO: 2, SEQ ID NO: 3, SEQ ID NO: 4, SEQ ID NO: 6, SEQ ID NO: 7 or SEQ ID NO: 8.

The term “hybridise” used here means hybridising under normal conditions such as are described in Sambrook et al. (Molecular Cloning. A laboratory manual, Cold Spring Harbor Laboratory Press, 2. Aufl., 1989) preferably under stringent conditions. Stringent hybridising conditions are for example: hybridising in 4×SSC at 65° C. and then washing many times in 0.1×SSC at 65° C. for a total of about 1 hour. Less stringent hybridising conditions are for example: hybridising in 4×SSC at 37° C. and then washing many times in 1×SSC at room temperature. The term “stringent hybridising conditions” used here can also mean: hybridising at 68° C. in 0.25 M sodium phosphate, pH 7.2, 7% SDS, 1 mM EDTA and 1% BSA for 16 hours and then washing twice with 2×SSC and 0.1% SDS at 68° C.

The invention also relates to DNA sequences which contain the coding region for an amino acid transporter or parts thereof, the RNA sequences corresponding to the DNA sequences and the mixed sequences composed of RNA and DNA nucleotides corresponding to the DNA sequences. In this situation, the DNA sequence comprises the nucleotide sequence of SEQ ID NO:1 or a nucleotide sequence complementary to the nucleotide sequence of SEQ ID NO:1, or hybridises with the nucleotide sequence of SEQ ID NO:1 or a nucleotide sequence complementary to the nucleotide sequence of SEQ ID NO:1. Alternatively the DNA sequence comprises the nucleotide sequence of SEQ ID NO: 2, SEQ ID NO: 3, SEQ ID NO: 4, SEQ ID NO: 5, SEQ ID NO: 6, SEQ ID NO: 7 or SEQ ID NO: 8 or a nucleotide sequence complementary to the nucleotide sequence of SEQ ID NO: 2, SEQ ID NO: 3, SEQ ID NO: 4, SEQ ID NO: 5, SEQ ID NO: 6, SEQ ID NO: 7 or SEQ ID NO: 8 or hybridises with the nucleotide sequence of SEQ ID NO: 2, SEQ ID NO: 3, SEQ ID NO: 4, SEQ ID NO: 5, SEQ ID NO: 6, SEQ ID NO: 7 or SEQ ID NO: 8 or a nucleotide sequence complementary to the nucleotide sequence of SEQ ID NO: 2, SEQ ID NO: 3, SEQ ID NO: 4, SEQ ID NO: 5, SEQ ID NO: 6, SEQ ID NO: 7 or SEQ ID NO: 8.

The invention further relates to vectors or mobile genetic elements which contain at least one DNA sequence and/or at least one RNA sequence corresponding to the DNA sequence and/or at least one mixed sequence composed of DNA and RNA nucleotides corresponding to the DNA sequence according to the present invention. Vectors or genetic elements which are suitable for introducing nucleotide sequences into host cells, for example, viruses, bacteriophages, cosmids, plasmids, artificial yeast chromosomes, T-DNA, transposons, insertion sequences etc., are well known to the person skilled in the art in the field of molecular cloning techniques.

The DNA sequence(s) and/or the RNA sequence(s) corresponding to the DNA sequence(s) and/or the mixed sequence(s) composed of DNA and RNA nucleotides corresponding to the DNA sequence(s) according to the invention is/are preferably contained in antisense orientation in the vector or the mobile genetic element.

In a further preferred embodiment the RNA sequence(s) is/are contained in an RNA double strand in the vector or the mobile genetic element. This makes it possible to achieve gene silencing of the endogenous amino acid transporter gene by RNA interference.

The invention further relates to eukaryotic or prokaryotic host cells which contain at least one DNA and/or at least one RNA sequence corresponding to this and/or at least one mixed sequence composed of DNA and RNA nucleotides corresponding to the DNA sequence according to the present invention, wherein the DNA, RNA and/or DNA-RNA mixed sequence(s) is/are preferably contained in antisense orientation in the host cell.

In a further preferred embodiment the RNA sequence(s) is/are contained in an RNA double strand in the eukaryotic or prokaryotic host cell.

The invention further relates to plants as well as parts or seeds of plants which are transformed with at least one DNA sequence and/or at least one RNA sequence corresponding to the DNA sequence and/or at least one mixed sequence composed of DNA and RNA nucleotides corresponding to the DNA sequence according to the present invention.

The plants can, for example, be plants of the genus Beta, preferably of the species Beta vulgaris. However, numerous other plants also come into consideration, for example, potatoes, tomatoes, sugar beet, tobacco, rape, Ricinius etc. By transforming the plants, plant parts or plant seeds with a DNA sequence, RNA sequence or DNA-RNA mixed sequence it is possible to obtain plants in which the expression of an amino acid transporter gene is suppressed for example, by means of co-suppression by interaction with a homologous ectopic (not sited at the normal position) gene sequence.

The transgenic plants, their parts or their seeds are preferably transformed with the DNA and/or RNA and/or DNA-RNA mixed sequence in antisense orientation. As a result of the transcription of the DNA sequence in antisense orientation, an mRNA is formed which hybridises with the naturally formed mRNA for the amino acid transporter at least partly so that a translation into the corresponding protein cannot take place. The introduction of an RNA sequence in antisense orientation results in an attachment of the RNA sequence onto the naturally formed mRNA so that the translation of the mRNA is also impeded in this case.

Further preferred are transgenic plants, their parts or their seeds which are transformed with an RNA sequence in an RNA double strand.

The present invention further relates to the use of DNA, RNA and/or DNA-RNA mixed sequences according to the invention for producing a transgenic plant cell or plant with enhanced expression of the coding region of the amino acid transporter compared with the wild type. The sequences according to the invention are used in this case in sense orientation. It is hereby possible for example to obtain plants which incorporate increased nitrogen compounds compared to plants of the wild type. In conjunction with suitable promoters it is also possible to influence the increased incorporation of nitrogen compounds in a tissue-specific fashion. In this way it is possible to produce plants, for example soya plants, whose harvestable parts have an enhanced content of organic nitrogen compounds compared with wild type plants.

The invention further relates to the use of a DNA sequence and/or a corresponding RNA sequence and/or a mixed sequence composed of DNA and RNA nucleotides corresponding to the DNA sequence according to the present invention in the antisense orientation to produce a transgenic plant cell or plant with reduced expression of the coding region of the amino acid transporter compared with the wild type. Compared with wild type plants, such transgenic plants have a reduced capability for incorporating organic nitrogen compounds in storage organs. The organic nitrogen compounds formed predominantly in the leaves are also not transported or only transported to a small extent into the storage organs in the senescence phase since the required amino acid transporter molecules are not present in sufficient quantity.

It has surprisingly been found that such plants have no impairments either in their growth or their development or in their other appearance. In addition, it has been found that the capability for incorporation of storage substances such as, for example, starch or saccharose is not adversely influenced in these plants. Rather, their storage capability is even increased compared with wild type plants.

The sequence protocol (according to WIPO-Standard St. 25) contains:
SEQ ID NO: 1: a nucleotide sequence of the coding region of the AAP1 gene of Beta vulgaris.
SEQ ID NO: 2: a nucleotide sequence of the coding region of the AAP6 gene of Beta vulgaris.
SEQ ID NO: 3: a nucleotide sequence of the coding region of the AAP2 gene of Beta vulgaris.
SEQ ID NO: 4: a nucleotide sequence of the coding region of the AAP3 gene of Beta vulgaris.
SEQ ID NO: 5: a nucleotide sequence of the coding region of the GAP1 gene of Beta vulgaris.
SEQ ID NO: 6: a nucleotide sequence of the coding region of a further AAP gene of Beta vulgaris.
SEQ ID NO: 7: a nucleotide sequence (BvProT-like) of the coding region of a further AAP gene of Beta vulgaris.
SEQ ID NO: 8: a nucleotide sequence (BvSV2-like) of the coding region of a further AAP gene of Beta vulgaris.

The amino acid transporter encoded by SEQ ID NO: 5 (BvGAP1) has proved to be a transporter with a broad amino acid spectrum. “GAP” thus also stands for “general amino acid permease”. BvGAP1 transports gamma amino butyric acid (GABA) particularly effectively. Acid amino acids such as aspartate are also transported particularly effectively. Citrulline, lysine and histidine are also transported.

The amino acid transporter encoded by SEQ ID NO: 7 (BvProT-like) and SEQ ID NO: 8 (BvSV2-like) specifically transports proline, citrulline and gamma amino butyric acid (GABA).

### BRIEF DESCRIPTION OF THE DRAWING

The invention is explained in detail in the following with reference to an exemplary embodiment but is not restricted to this: in the figures:

FIG. 1: shows the expression of BvAAP genes (BvAAP1, BvAAP2 and BvAAP6) in various stages of senescence of leaves as well as in five- and six-month-old storage roots of Beta vulgaris, determined by Northern hybridisation. 18s rRNA was used as the control. The meanings are as follows:

leaf RNA: RNA from leaves

beet RNA: RNA from storage roots

### DETAILED DESCRIPTION OF THE INVENTION

### EXAMPLE

From senescent leaves and storage root material of sugar beet (Beta vulgaris) RT-PCR (reverse transcription with subsequent polymerase chain reaction) was used to isolate and sequence three nucleotide sequences which comprise the coding region of amino acid transporters (BvAAP1 and BvAAP2, BvAAP6), or parts thereof. cDNA obtained from the nucleotide sequences was cloned into the yeast expression vector pDR196.

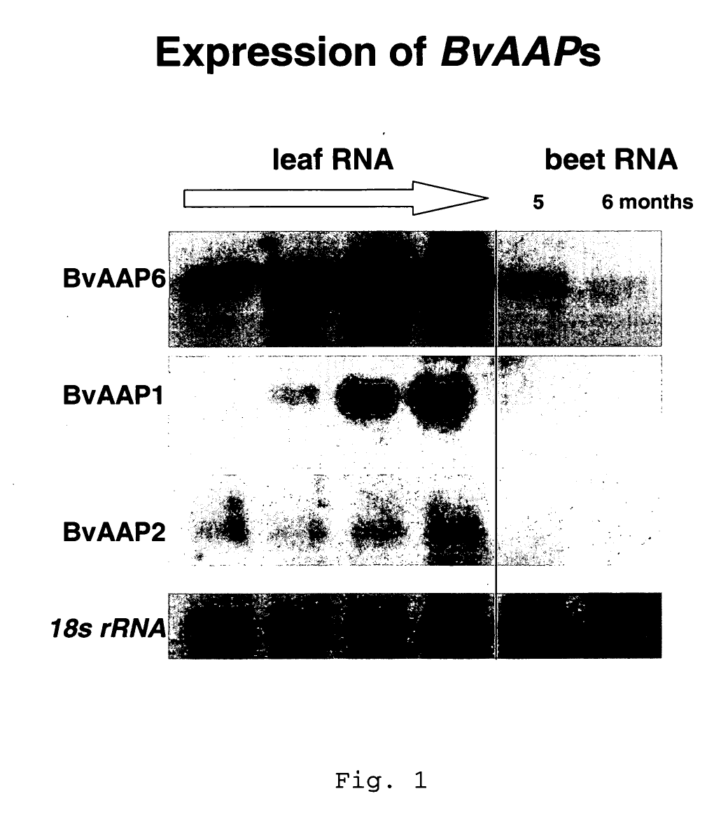

Expression studies on total RNA level revealed that the expression of BvAAP6 increases strongly during senescence (FIG. 1). The transcript quantity of BvAAP1 also increases during senescence. The transcript quantity of BvAAP2 on the other hand barely changes. As a result, it becomes clear that the two amino acid transporters BvAAP1 and BvAAP6 in particular play a decisive role in the transfer of nitrogen compounds from ageing leaves of the sugar beets into the storage roots. The results also show that BvAAP2 is also involved in the distribution of amino acids.

The vector pBin19 and derivatives hereof are suitable, for example, as vectors for the transformation of sugar beet. A cloning in antisense orientation, for example, under control of the CaMV-35S promoter is also possible here.

1. A method for producing a transgenic plant in which the capability for incorporating undesired nitrogen compounds in the harvest organs is reduced compared with the wild type, comprising the steps of
introducing at least one DNA sequence and/or at least one RNA sequence corresponding to the DNA sequence and/or a mixed sequence composed of DNA and RNA nucleotides corresponding to the DNA sequence with the coding region for an amino acid transporter or parts thereof into a plant cell, wherein the DNA and/or RNA sequence and/or mixed sequence is used in sense or antisense orientation and the expression of an endogenous amino acid transporter gene is prevented or reduced, and
regenerating a plant from this plant cell, wherein the DNA and/or RNA sequence is a sequence from Beta vulgaris.

2. The method according to claim 1, wherein the DNA sequence
a) comprises the nucleotide sequence of SEQ ID NO: 5 or a nucleotide sequence complementary to the nucleotide sequence of SEQ ID NO: 5 or hybridizes with the nucleotide sequence of SEQ ID NO: 5 or a nucleotide sequence complementary to the nucleotide sequence of SEQ ID NO: 5, and/or
b) comprises the nucleotide sequence of SEQ ID NO: 1 or a nucleotide sequence complementary to the nucleotide sequence of SEQ ID NO: 1 or hybridizes with the nucleotide sequence of SEQ ID NO: 1 or a nucleotide sequence complementary to the nucleotide sequence of SEQ ID NO: 1, and/or
c) comprises the nucleotide sequence of SEQ ID NO: 2 or a nucleotide sequence complementary to the nucleotide sequence of SEQ ID NO: 2 or hybridizes with the nucleotide sequence of SEQ ID NO: 2 or a nucleotide sequence complementary to the nucleotide sequence of SEQ ID NO: 2, and/or
d) comprises the nucleotide sequence of SEQ ID NO: 3 or a nucleotide sequence complementary to the nucleotide sequence of SEQ ID NO: 3 or hybridizes with the nucleotide sequence of SEQ ID NO: 3 or a nucleotide sequence complementary to the nucleotide sequence of SEQ ID NO: 3, and/or
e) comprises the nucleotide sequence of SEQ ID NO: 4 or a nucleotide sequence complementary to the nucleotide sequence of SEQ ID NO: 4 or hybridizes with the nucleotide sequence of SEQ ID NO: 4 or a nucleotide sequence complementary to the nucleotide sequence of SEQ ID NO: 4, and/or
f) comprises the nucleotide sequence of SEQ ID NO: 6 or a nucleotide sequence complementary to the nucleotide sequence of SEQ ID NO: 6 or hybridizes with the nucleotide sequence of SEQ ID NO: 6 or a nucleotide sequence complementary to the nucleotide sequence of SEQ ID NO: 6.
g) comprises the nucleotide sequence of SEQ ID NO: 7 or a nucleotide sequence complementary to the nucleotide sequence of SEQ ID NO: 7 or hybridizes with the nucleotide sequence of SEQ ID NO: 7 or a nucleotide sequence complementary to the nucleotide sequence of SEQ ID NO: 7.
h) comprises the nucleotide sequence of SEQ ID NO: 8 or a nucleotide sequence complementary to the nucleotide sequence of SEQ ID NO: 8 or hybridizes with the nucleotide sequence of SEQ ID NO: 8 or a nucleotide sequence complementary to the nucleotide sequence of SEQ ID NO: 8.

3. A DNA sequence which contains the coding region for an amino acid transporter or an RNA sequence corresponding to the DNA sequence, or a mixed sequence composed of RNA and DNA nucleotides corresponding to the DNA sequence, wherein the DNA sequence
a) comprises the nucleotide sequence of SEQ ID NO: 5 or a nucleotide sequence complementary to the nucleotide sequence of SEQ ID NO: 5 or hybridizes with the nucleotide sequence of SEQ ID NO: 5 or a nucleotide sequence complementary to the nucleotide sequence of SEQ ID NO: 5, or
b) comprises the nucleotide sequence of SEQ ID NO: 1 or a nucleotide sequence complementary to the nucleotide sequence of SEQ ID NO: 1 or hybridizes with the nucleotide sequence of SEQ ID NO: 1 or a nucleotide sequence complementary to the nucleotide sequence of SEQ ID NO: 1, or
c) comprises the nucleotide sequence of SEQ ID NO: 2 or a nucleotide sequence complementary to the nucleotide sequence of SEQ ID NO: 2 or hybridizes with the nucleotide sequence of SEQ ID NO: 2 or a nucleotide sequence complementary to the nucleotide sequence of SEQ ID NO: 2, or
d) comprises the nucleotide sequence of SEQ ID NO: 3 or a nucleotide sequence complementary to the nucleotide sequence of SEQ ID NO: 3 or hybridizes with the nucleotide sequence of SEQ ID NO: 3 or a nucleotide sequence complementary to the nucleotide sequence of SEQ ID NO: 3, or
e) comprises the nucleotide sequence of SEQ ID NO: 4 or a nucleotide sequence complementary to the nucleotide sequence of SEQ ID NO: 4 or hybridizes with the nucleotide sequence of SEQ ID NO: 4 or a nucleotide sequence complementary to the nucleotide sequence of SEQ ID NO: 4, or
f) comprises the nucleotide sequence of SEQ ID NO: 6 or a nucleotide sequence complementary to the nucleotide sequence of SEQ ID NO: 6 or hybridizes with the nucleotide sequence of SEQ ID NO: 6 or a nucleotide sequence complementary to the nucleotide sequence of SEQ ID NO: 6.
g) comprises the nucleotide sequence of SEQ ID NO: 7 or a nucleotide sequence complementary to the nucleotide sequence of SEQ ID NO: 7 or hybridizes with the nucleotide sequence of SEQ ID NO: 7 or a nucleotide sequence complementary to the nucleotide sequence of SEQ ID NO: 7.
h) comprises the nucleotide sequence of SEQ ID NO: 8 or a nucleotide sequence complementary to the nucleotide sequence of SEQ ID NO: 8 or hybridizes with the nucleotide sequence of SEQ ID NO: 8 or a nucleotide sequence complementary to the nucleotide sequence of SEQ ID NO: 8.

4. A vector or mobile genetic element, containing at least one DNA sequence according to claim 3 and/or at least one RNA sequence corresponding to the DNA sequence and/or at least one mixed sequence composed of DNA and RNA nucleotides.

5. The vector or mobile genetic element according to claim 4, wherein the DNA sequence and/or the RNA sequence corresponding to the DNA sequence and/or the mixed sequence is contained in antisense orientation.

6. The vector or mobile genetic element according to claim 4, wherein the RNA sequence is contained in an RNA double strand.

7. A eukaryotic or prokaryotic host cell containing at least one DNA sequence according to claim 3 and/or one RNA sequence corresponding to the DNA sequence and/or at least one mixed sequence composed of DNA and RNA nucleotides.

8. The eukaryotic or prokaryotic host cell according to claim 7, wherein the DNA sequence and/or the RNA sequence corresponding to the DNA sequence and/or the mixed sequence is contained in antisense orientation.

9. The eukaryotic or prokaryotic host cell according to claim 7, wherein the RNA sequence is contained in an RNA double strand.

10. A plant or parts thereof transformed with at least one DNA sequence according to claim 3 and/or at least one RNA sequence corresponding to the DNA sequence and/or at least one mixed sequence composed of DNA and RNA nucleotides.

11. The transgenic plant according to claim 10, whereini the plant is transformed with the DNA sequence and/or the RNA sequence and/or the mixed sequence in antisense orientation.

12. The transgenic plant according to claim 10, wherein the RNA sequence is contained in an RNA double strand.

13. Seeds of plants according to claim 10.

14. A method for producing a transgenic plant cell or plant with an enhanced expression of the coding region of the amino acid transporter compared with the wild type, comprising transforming a plant cell or plant with a DNA sequence which contains the coding region for an amino acid transporter and/or an RNA sequence corresponding to the DNA sequence, or a mixed sequence composed of RNA and DNA nucleotides corresponding to the DNA sequence, wherein the DNA sequence
a) comprises the nucleotide sequence of SEQ ID NO: 5 or a nucleotide sequence complementary to the nucleotide sequence of SEQ ID NO: 5 or hybridizes with the nucleotide sequence of SEQ ID NO: 5 or a nucleotide sequence complementary to the nucleotide sequence of SEQ ID NO: 5, or
b) comprises the nucleotide sequence of SEQ ID NO: 1 or a nucleotide sequence complementary to the nucleotide sequence of SEQ ID NO: 1 or hybridizes with the nucleotide sequence of SEQ ID NO: 1 or a nucleotide sequence complementary to the nucleotide sequence of SEQ ID NO: 1, or
c) comprises the nucleotide sequence of SEQ ID NO: 2 or a nucleotide sequence complementary to the nucleotide sequence of SEQ ID NO: 2 or hybridizes with the nucleotide sequence of SEQ ID NO: 2 or a nucleotide sequence complementary to the nucleotide sequence of SEQ ID NO: 2, or
d) comprises the nucleotide sequence of SEQ ID NO: 3 or a nucleotide sequence complementary to the nucleotide sequence of SEQ ID NO: 3 or hybridizes with the nucleotide sequence of SEQ ID NO: 3 or a nucleotide sequence complementary to the nucleotide sequence of SEQ ID NO: 3, or
e) comprises the nucleotide sequence of SEQ ID NO: 4 or a nucleotide sequence complementary to the nucleotide sequence of SEQ ID NO: 4 or hybridizes with the nucleotide sequence of SEQ ID NO: 4 or a nucleotide sequence complementary to the nucleotide sequence of SEQ ID NO: 4, or
f) comprises the nucleotide sequence of SEQ ID NO: 6 or a nucleotide sequence complementary to the nucleotide sequence of SEQ ID NO: 6 or hybridizes with the nucleotide sequence of SEQ ID NO: 6 or a nucleotide sequence complementary to the nucleotide sequence of SEQ ID NO: 6,
g) comprises the nucleotide sequence of SEQ ID NO: 7 or a nucleotide sequence complementary to the nucleotide sequence of SEQ ID NO: 7 or hybridizes with the nucleotide sequence of SEQ ID NO: 7 or a nucleotide sequence complementary to the nucleotide sequence of SEQ ID NO: 7,
h) comprises the nucleotide sequence of SEQ ID NO: 8 or a nucleotide sequence complementary to the nucleotide sequence of SEQ ID NO: 8 or hybridizes with the nucleotide sequence of SEQ ID NO: 8 or a nucleotide sequence complementary to the nucleotide sequence of SEQ ID NO: 8.

15. A method for producing a transgenic plant cell or plant with an enhanced expression of the coding region of the amino acid transporter compared with the wild type, comprising transforming a plant cell or plant with a DNA sequence which contains the coding region for an amino acid transporter and/or an RNA sequence corresponding to the DNA sequence, or a mixed sequence composed of RNA and DNA nucleotides corresponding to the DNA sequence, wherein the DNA sequence
a) comprises the nucleotide sequence of SEQ ID NO: 5 or a nucleotide sequence complementary to the nucleotide sequence of SEQ ID NO: 5 or hybridizes with the nucleotide sequence of SEQ ID NO: 5 or a nucleotide sequence complementary to the nucleotide sequence of SEQ ID NO: 5, or
b) comprises the nucleotide sequence of SEQ ID NO: 1 or a nucleotide sequence complementary to the nucleotide sequence of SEQ ID NO: 1 or hybridizes with the nucleotide sequence of SEQ ID NO: 1 or a nucleotide sequence complementary to the nucleotide sequence of SEQ ID NO: 1, or
c) comprises the nucleotide sequence of SEQ ID NO: 2 or a nucleotide sequence complementary to the nucleotide sequence of SEQ ID NO: 2 or hybridizes with the nucleotide sequence of SEQ ID NO: 2 or a nucleotide sequence complementary to the nucleotide sequence of SEQ ID NO: 2, or
d) comprises the nucleotide sequence of SEQ ID NO: 3 or a nucleotide sequence complementary to the nucleotide sequence of SEQ ID NO: 3 or hybridizes with the nucleotide sequence of SEQ ID NO: 3 or a nucleotide sequence complementary to the nucleotide sequence of SEQ ID NO: 3, or
e) comprises the nucleotide sequence of SEQ ID NO: 4 or a nucleotide sequence complementary to the nucleotide sequence of SEQ ID NO: 4 or hybridizes with the nucleotide sequence of SEQ ID NO: 4 or a nucleotide sequence complementary to the nucleotide sequence of SEQ ID NO: 4, or
f) comprises the nucleotide sequence of SEQ ID NO: 6 or a nucleotide sequence complementary to the nucleotide sequence of SEQ ID NO: 6 or hybridizes with the nucleotide sequence of SEQ ID NO: 6 or a nucleotide sequence complementary to the nucleotide sequence of SEQ ID NO: 6.
g) comprises the nucleotide sequence of SEQ ID NO: 7 or a nucleotide sequence complementary to the nucleotide sequence of SEQ ID NO: 7 or hybridizes with the nucleotide sequence of SEQ ID NO: 7 or a nucleotide sequence complementary to the nucleotide sequence of SEQ ID NO: 7,
h) comprises the nucleotide sequence of SEQ ID NO: 8 or a nucleotide sequence complementary to the nucleotide sequence of SEQ ID NO: 8 or hybridizes with the nucleotide sequence of SEQ ID NO: 8 or a nucleotide sequence complementary to the nucleotide sequence of SEQ ID NO: 8.

16. A method according to claim 15, wherein said transgenic plant is a transgenic plant with a reduced capability for incorporating organic nitrogen compounds compared with the wild type, especially glutamine, asparagine, citrulline, gamma amino butyric acid and auxine, in storage organs.

17. A method according to claim 15, wherein said transgenic plant is a transgenic plant with an elevated storage capacity for storage substances compared with the wild type.


SEQUENCE LISTING (8 sequences)

SEQ ID NO: 1 - DNA, 674 bp, Beta vulgaris
gcgcatattatcacagccgttattgggtcaggggtgttgtcattggcgtgggcaatagcacagctaggatgggcggttggtccggctgtattggtggctttctcgttcatcacatacatgacttcgacgtttctcgccgacggttatcgctcacctgatcctgttactggcaagaggaattacacttacatggaagttgtccgagccaatttaggtggcaggaaagttcaactatgtggcttagctcaatacgtgaatttggtaggcgtctcaattggatacacaattactgcctctattagcatggtagcggtaaaaaggtcaaattgtttccacaagtacgggcaccatgtgaagtgcaacacatcaaacaatccgttcatgataatatttgggtgcatccaaattctgctcagtcaaatcccaaactttcacgagctttcttggctttctatggtggcagcaattatgtcttttacctatgcttctattggcctcgctctctccatcgccaaagttgtaggtgggggaatcgcaagaacatcattaacaggggtaacggttggagtggacgtgacagccgcggacaaggtgtggaggtgtttccaagccataggcgatattgcctttgcctatgcttattctaatgtcctcattgagatccaagacacagt

SEQ ID NO: 2 - DNA, 1870 bp, Beta vulgaris
ccaacaccacactattaagtactactatcatttttcaactatagcttcagttatcctctcatccgagtgcgtgagttagagagataacacacaaaaaaaccatggtctctcacaacaacggcaccaccaccaccaccaccaacaataatatgtacatagaacaaggtggccatggtggaaatgtccatgatttcgacaaaaacctcgacgacgatggccgtcacaaaagaactgggacggcgttgacggcaagtgcacacataataacggcggtgatagggtcaggggtgttgtcattggcgtgggcaatagcacagctaggatgggcggttggtccggctgtattggtggctttctcgttcatcacatacatgacttcgacgtttctcgccgacggttatcgctcacctgatcctgttactggcaagaggaattacacttacatggaagttgtccgagccaatttaggtggcaggaaagttcaactatgtggcttagctcaatacgtgaatttggtaggcgtctcaattggatacacaattactgcctctattagcatggtagcggtaaaaaggtcaaattgtttccacaagtacgggcaccatgtgaagtgcaacacatcaaacaatccgttcatgataatatttgggtgcatccaaattctgctcagtcaaatcccaaactttcacgagctttcttggctttctatggtggcagcaattatgtcttttacctatgcttctattggcctcgctctctccatcgccaaagttgtaggtgggggaatcgcaagaacatcattaacaggggtaacggttggagtggacgtgacagccgcagacaaggtgtggaggtgtttccaagccataggcgatattgcctttgcctatgcttattctaatgtcctcatcgagatacaagacacactaaaatcatcccctccagaaaacaaagtaatgaagaaggcatccctggttggggtatccaccaccagcctgttttacatcctttgtggttgctttggttatgccgcgtttggtaatgaagcccctggaaacttcctcactggatttggattttacgagcccttttggcttgtcgattttgccaacgtttgcattgcagttcatctagttggtgcttaccaggttttcacccaacccgtgtttggcttcgtcgagtcatggtcaaagaagcattggccggaaaacaaattcataacaagggatcacccaatcgagatcccttttggcaatgggatctactacttcaactggtttagactagtatggaggacatcatatgtgatagtaacagcaattctagctatgttattcccattcttcaacgatttccttgggttattaggatcactatcattttggccactgactgtgtttttccccgtcgagatgcacatctcacaagcaaaggtgcccaagtactcgtcaaagtggatagctctaaaaacacttagctatgcttgcttggtagtgtcattaatcgcggcagctgggtcgattcagggacttgttggttctgtaaagaaatacaagccctttcaggcacaagaatgagtctcttttaactcctttgatcctatttgttgtgaaatatttagcaaaaatgtagtgcgacattccagtaatgtggtaaaagttttagatctttgatctcgttcagaacatctaatcctctagcataaggtagtgatgtaattgaaaaaattgcaaaccccttgtgaagctttgttgtgtaattagtggcactaaattagctagatgattggaaacttaaatacaactggttttctaatgtcacaattatggatgaaaatcctttacatttgatcaaaaaaaaaaaaaaaaaa

SEQ ID NO: 3 - DNA, 2004 bp, Beta vulgaris
agcttagcagcatttttctagcatttttgttttggcattgaatacatagtcacctaccaagctacacaagtgcaaataatcacttcattcttaagctcatctagctaatttttagctctcataaacaccctattataaagatcacaccttatttacaccattaatctccttaccttattctctaattgtgcataatttttttgctctcctttcctttcaagatgggtgatggtgctgccaaaataaacaaccaccatgtcttcgatgtctccttcgacgtgctaggacaaaatccctccaaatgcttcgacgatgatggtcgtctcaaaagaacagggactgtttggactgcaagtgcacatattataacagctgttataggctctggagtgttgtccttagcatgggctatagctcagcttggctggattgctgggccagcagtgatgttcttgttctcctttgtaatctactatacttctactcttcttgctgactgctatcgttccggtgatccgaattctggcaagagaaactacacgtacatggatgctgttagatctaaccttggtggtggaaaggtcaaattctgtggaatgattcaatacttgaacctctttggtgttgcaataggatacacaattgcagcatctattagcatgatggcaattaaaagatcaaactgcttccatgatagtggtggaaagaacccatgtcacatgtcaagcaacccatatatgattgcttttggaattgcagaaatcttattttctcagattcctgactttgatcaaatatggtggctctccattgttgcagctgttatgtctttcacttactctgccattggtcttgccttaggaattgttcaagtagcagctaacaaaactttcaagggtagtttcactggaattagcataggttcagtaactgaaacacagaagatatggaggagtttccaagcccttggtgatattgcctttgcttactctttctcaatcatccttattgagatccaggacacagttagatcccctccggcagaatccaagacaatgaagaaagcctcattcataagcattgcggttacaacagtgttttacatgctgtgtggatgcatgggttatgcggcgtttggtgatctagcaccagggaatctcttaactggttttgggttctacaatccatattggctcctagacatagccaatgtagccatagtaattcacttagttggagcataccaagtttactgtcaacccctctttgcatttgttgaaaaatgggcatctcagaaatggcctgacagtgatttcatccaagagaaatgaagattagaattccgggtattggtccatacaacttaaactcgtttaggttggtgtggagaacgacatttgtgataacaacgacgattatatcaatgctgctacctttcttcaatgacatagtaggattacttggtgcacttggattttggcctttaactgtgtattttccaattgagatgtacattgctcaaaagaaggtaccaaagtggagtacaaagtggttgtgtcttcaaatgttgagcatggcttgtctcgtgatttcgattgctgctgctgcaggatcgattgctggtgtagctcttgatctaaaggtgtaccgtccattcaaaactagctactgatcgaatcatggaagttatcattagtacttggatcaacttgagtaggagaatagtaaaaatctatttgtatttcaatatatattcatggaggaatcatgttgatcaatcatgggtggtacagttgtattttctttttaagtattctaaatgtgtatttcttcccttgttggtgctgtaacaaaaagtttgctagggttcataagaaatggaggaaccctagatttcatgtgtattttagtgtttaatttgtaattgtttcagaattgagtttctactctattcttttgtcaatccattagttttctctagtgtaaaaaaaaaaaaaaaaaaaa

SEQ ID NO: 4 - DNA, 2079 bp, Beta vulgaris
ctgtagttacttcccttagtacagcaccagactacatacacatcacacagtacaacagtagcgtaggagttgagggaagtactactcatatataataggttgggtcaaaattacttgacaacagaatagtaagactatctaatttagtcggtgaagtgaagaagatggaattaggaagcaagcagctaaagaatgaatacaacttcgataatagtaatcaatccttccatttgacgacgtttgatgtcgactccgccaccggttccggctccaaattatttgacgatgatggccgtctcaagagaagagggactatgtggacagcaagtgcacatataataacagcagtgattgggtcaggggtgttatcccttgcttgggctacagcccaacttggatgggtcgctggcccaattgtgatgttgctattttccattgtaacttactacacttctgtactccttgctgactgttaccgctccggggaccctgtctctggcaagaggaattacacctacatggacgccgttcaagctaatcttggcggccttcaagtgaaaatatgtggactaattcagtaccttaacctctttggagttgcaatcggttataccatagcagcgtctataagcatgacggctataaagaggtcgaattgtttccatgagaaaggagagaaaagcaaaatgcgacatatcaaccaaatccttacatgattgcctttggtggttgctgagataatcttctctcaaatcccagattttgatcaaaatctggtggctatcaattgttgcggcggtcatgtctttcacctattccaccattggtcttggtctgggtattgctaaagttgctgtgaacggacaagtcaagggcagtctgactggtattagtattggcgtggtgacacaaaccgataagatatggaggagcttccaagcacttggtgatattgcatttgcatattcttactctatgatcctcattgagattcaagacactgtaagatcccccccatccgaatcaaagacaatgaggaaggctacacttataagcgtagtagtgacaaccttcttttacatgttatgtgggtgctttggttatgcagcatttggggacttgtcacccgggaatcttctaaccgggtttgggttctacaacccattttggctggttgacatagccaacgctgccatcgttatccacctgattggtgcctaccaagtttactgccaacctctctatgctttcattgagaaaatagccagtgaaaacttccccaacagtgacttcatcaccaaagaaatcttgatcccacttccaggtggttacaaaccttacagacttaatatgttcaggctagtttggaggacaatctttgtgatagttacgaccgtaatctcaatgctcttacccttcttcaacgacgtagttggattgcttggagcactcggattttggccattgactgtatatttcccagtagagatgtatatctcgcagaaaaagattcccaaatggagtactaaatggatttgccttcagatgcttagttttgcttgcctcatcatcaccatcgccgctgctgctggatcagttgctggggtgattcttgatcttaagtcttataaacctttcaagaccacttactgattggtattctttgcgtacattacaatttacatggctagctattagctaacaaaatcaatggttacgacaagttatttttttctttctgtggagagaaaggcaccagtgcccacaaaaggcgggccgagctagggtatatatgatataatgctccatgtagttgttgccccttcggttaatcatttgtatgtatctctatgttacagctccatggacattgtattacctttttaatttacaggtctcatcacattttactaatgcacctttgttatagaatccccactcctggccctaagcccctaattaacaaccatcttatggtaagggaatggaattagctgaattattagcttttttctaccagattggtatgtaataaagctccagccacatcaagaatataaaaaaaaaaaaaaaaaaaaa

SEQ ID NO: 5 - DNA, 1733 bp, Beta vulgaris
ctactatatagtaagtatattgctgattattagctggagaaatcagcaggagattaattagcttgaaaagttggattatttatatctgttactactataaagctaagttgatctgatttttggagatttattatcaagcaaataaaatgggtactcaagctcctgctgaacactataatacaccggatattgaagcaaagttgcaaaaacaaaaggacattgatgcttggcttccgatcacttcttcaagaaatgcgaaatggtggtattctgctttccacaatgtcactgccatggttggtgctggagtccttagtcttccatatgctatgtctaatctaggatgggggccaggagtggttatattagtattatcatggatcattacattgtacactctatggcaaatggtggagatgcacgagatggtgccagggaagcgatttgatcggtaccatgagttgggacaatatgcctttggtgagaaactaggcttgtggattgtggtgcctcaacaacttatcgtcgaagttggtgttaacattgtttacatggtcactggaggcaggtccctcgaaaaggtccatgatattatatgtgccaataaaacaagttgtacccccatgaaaaccacctacttcatcatgatcttctcttctgtccacttcgtcctttctcacctccctaacttcaactccatcgcgggcgtgtccttggctgccgcggtcatgtctctcagctactccacaattgcatgggcaacatcactaggcaagggtgtacaaccacatgttacctacacaagtaggcaccacagcgacgtagggaagacattcgggttcttcaatgctctcggcgatgtggctttcgcatacgcaggccacaatgttgtgctcgaaattcaagcaaccatcccttccaccccagagaagccttccaagggacctatgtggaagggtgtcatcgtcgcctacatcgtcgtcgccttgtgctacttccccgttgcccttattggttactatgtttttggtaacactgttagagataacatccttatttcccttgagaaacctggttggcttatagctatggccaacattttcgttgttattcacgtcattggaagctatcagatatatgccatgccagtgtttgacatgatcgaaaccgtgctagtgaagaaaattcgctttaagcctacttggtaccttagatttatcgctcgtaacatctatgttgcatttacaatgtttgttgccattacattccctttctttggtggacttcttggattcttcggaggattcgctttcgctccaactacatactttcttccttgcataatgtggcttgctatctacaaacccaagagatttagcctctcatggtgcgccaactggatttgcattgttctgggagttatattgatggtgttagcgcccataggagggctaagacagatcataattgactcaaaaaactacacgttctactcttaagaagcttcctaaattcgcggtttgaagaattagcaatggagtaatttgttttgataatgggggatatgatgaaaatcctacattatcttagtgcaaattaactagggtacagtcagtcatacattttctgaaacctctactattctcttcgttaattcaaaagtgttttttgtgtgcataatagaaatatttctgctctgtgtttccaatcaaaaaaaaaaaaaaaaaaaaaaa

SEQ ID NO: 6 - DNA, 1548 bp, Beta vulgaris
acttcgcgtgcctttgcaattctctttgttgtgcttcttgttatcctcccattggtcttgtttcgtcgtgtagattcacttaggtacacttcgttactggccattcttcttgccttggtgtttgttgtcatatcttcagtattggcaatagaagcgatgttggaaggaaagacacagaaccttcggcttgctccagatttctcccgtcaaggaaccttcctcagtctttttaccaccattccagtttttatgactgcttttggataccaaatcaatgttcatccaattagagcggaacttggagaaccttctgacatgagaatggctatcaggatatcactcataatatgtgctctcttttatttctccatcggtttctttggctatctattatttggggattctattgatgctgacatgctagtgaacttcgatcagattggtcatgatactgtggttggtttagtaattaactctattgttcgattaagctatgcagttcacctaatgctggtgtttcctttaataaattactgtctcagagccaacatagacgagctcattttccccaataagcctgacttagccactaatactacaagattcatatgcatcacatgtataccactcgcccttgcgtatgtaattgcaatctggataccaagcatatggtattgcttccagatattgggttcgacaacagttgtttgcctcatactcatatttccatcatcaattattttaagggatatacatgggatctctacattaaaggacaacgtgattgcagtaatgcttatacttatcgcagtatttgccagcacaatcgctatatggaccaatttgtccactccagcagggagtaaagcttagcggtttgtactttgcacacactattgcccacattttagcgggtttcatagtccatcatgatttatgtatgaactccagttttagctgcagttttgatcaggtggattgtctttctatacgatcaggcattgtctttgcttctacgcctaaattgctcttccaaattcacaaatcctctcttggtggttgaatcaatcaatgtatatactacatgtaaatatgcttttctggaccactaacaagagaagattatcaatttccattcgttgaagtctcgttcgtagcattcttcatcttgtgttaaagatttcatatgttttaatgaaactttaatgtggaggatacaatgtacgtcgttgtttgttggctgtatgaaatttgctgaagtgaaagtggcgcggcccaaagtgcaaaggggcttgtcttgagagaaaacgttcttagatttgttctttccttgtatattcaaaaaaaaagaaaaaggagaaggaaagagtgagaggaacagtatgtaatttgtaagctttgtaatgttacacacttacactcttatagtcttatctatgttctacacttctacttgaatgtattcagtgcctaggttctttggtcttagagtaaggccaaaagaaattatgaagtagaagattacttttaaaaaaaaaaaaaaaaaaa

SEQ ID NO: 7 - DNA, 1731 bp, Beta vulgaris
gaaaattttgagtgagaggcaaaatgagtaaccgagaagcttttaatatcgaaacaaactccataaaagatgaccaaatcctcgaagagggaccagcttcagctcacgctattggcaatgattcatggcagcaggtcagcttgttgctcgtaacgagcttcaattgtggatggatattgagcttttcagatcttatgttggtgcccctgggttgggtatggggtatcatctgcttacttcttgttgggttctttagttgctactcaagctgggtagcagcaggttttcacttcatcgatggccagagattcatcagatacagagacctcatgggagttctatttggaaagaaaatgttctacaccacatggattttacaattttcaacctttattttgggaaatatgggtttcatccttcttgggggaaaagcactgcaggagataaatctggtgttcagcgacacaaccatgaggctacagtatttcatcattatcacaggaacaacatactttattttttcttttgttgtcccaaacctgtctgccatgagagtgtggttaggattgtcggtaattctaaccttcgcttatataggagtccttcttgtgacaacaataaacgaaggcaaacttataggcaaatctagtagacatagagactatgacgttaagggaagcaaggtggataggatatttaatggcttcggagcaatatcagccatcattgtatgcaacaattcaggcatggtaccagaaatacagtcaaccctccggaagccagctgtcaaaaacttgcgaagagctttgtatgtacaatatactcttggcctcataatatattatggtgttagtatagctggatactgggcttatggttcggaagtgccagattatctccctaaggcactaagcgggccaaaatgggcgaaggtcctgatcaacttagcagtctttctgcaaaatgtcatttcacaacatatgtttttccaacctgttcatgaggccctagataccaagttcctgaaagttgacgagggtatatactcgagagaaaatttgaaaaggcgattcttcctacgagcactcctatttataggaaatactatcgtgacagcggccataccttttatgggagatttcattaaccttttgggatcattcacactagtgacattgacttttatctttccgagcatgatatttatcaaggttaagggaaagacagctaggattgtgcacacagcatggcattggactatcgttcttgtttttaccctgcttgcaatggcaacaacaatttctgcagtccgaaaagtgattaaagatgttacaaactaccatttattttccaataatcaatgatatattgaagtgcaaataaacagccacattctctactcaaggtcataatgttcctatgactagcttccacggtttctttttatgtaaaagggcaaacaaacctttcggctgcacttcagaggattctttatgcaatatgcaaaaagtgacatatccagcagtgcttaacaaaagaaaaatgttcaagtaacaatggatctacaaagtgaagcagttgaaggctatcaacataaattagtgaagggccatgaaaagttcgatagttacttgtaagctgtatagatatgcttggggaattatatatctttccagttcacagaaaaaataatcaggtaacagaaaaaaaaaaaaaaaaaaaaaaaaaaaa

SEQ ID NO: 8 - DNA, 1801 bp, Beta vulgaris
cattatcatttatcaaatattatctcatttctaagaaaaagaaagtggtcttgttaattacagattaattatggcggatactggaggattaatatatagtgtagatgacgctatatcgaagatggggtttggaaaattacagtatatggtgttagcatatgctggaatggggtggatatcagaagctatggagatgatgatactttcatttgttggaccagcagttacatttgagtggaatcttacttctaaacaggaaagtttgatcacaactgtggtttttgctggcatgcttgttggtgcttattcttggggacttgtttctgatatctatggaagaaggaaaggttatgtgttttcggcagtagtaacttgtgcggctggattactgagtgccgcttcaccaaattacgtagtattgttaatatctcgttgtttggtaggcgtgggtcttggagctggcccagttctactcacctggtttatagagtttgtacctgcatctaaaaggggtatctggatggtccttttccaagctttttggacagtcggatccgttagtgaagctgccttagcatgggttgtaatgccgaaattgggatggagatggttgttgggtttatctgttcttccttcatttatcttgcttgtgttctatttcattactccagagtcgccaagatacttgtgcttgaaaggtagaaagaaagaagcacttcaagtccttgagcaaatatcccgaattaatggtgaagaattacctcctggtgttcttgttagtgacatggaaaaagaagagaagaataataatatactaaaaggagttacagatgaagaagaagttgctcctaaatggaaggattcagaaatgagcttaacaaaatcagttttagttcttctgtctcccaaattagcccgtacaactttactcctatggttggtgttctttggaaatgcgtttgcgtactatggtctcgtcttgcttacaactcaactgaacaacaagaacaatgcatgcaattctaaaaattcccctagttcaaaaagttctaagttagatattgattatcgagatgtcttcatcactagttttgctgaacttcctggccttgttattgctggtatcttaattgataaacttggtcgtaagtattcgatggcgatcatgtttgtagcatgttcaatatttttgttcccattggtgtctcatcggtctggtacggttacaacagttctcatgtttggtgctcgagcatccatcatgggaaccttcaccattgccttcattttcgctccagagatatacccaacatcagtaagaaatactggattcggaacagcgagttcaatggcaagagtaggtggaatggtgagcccatatgtagctgtggctttagtacaagcttgccatcagatggcggcaatcctattatttgcaggaatggtgttagctgctgcagtagctgttgtacttattccacatgaaacaaaggggcgtgagcttactgaaagcatcatgagtaataagaatgatattaaatcaagcaaagaagtttctctacaaaaaccaactagttcaaatccgaaggccatgacataagaagttgctttaaacagacaactaattcgaataacggttaacggtttaaattatcatgtttgttaattgagatcaagattatagaaacaaaagtgttgtttcgttttcaaacattttaggctatttttccggtgtccagatgcatacgtaattactaagtggttttatatgatcttaaaaaaaaaaaaaaaaaaaaaaaaa